(19) United States
(12) Patent Application Publication
(10) Pub. No.: US 2008/0275524 A1
(43) Pub. Date: Nov. 6, 2008

(54) Implantable Electrode Arrangement

(75) Inventors: John Furness, Victoria (AU); Zoran Milijasevic, New South Wales (AU); Jim Patrick, New South Wales (AU)

Correspondence Address: JONES DAY, 222 EAST 41ST ST, NEW YORK, NY, 10017, US

(73) Assignee: Continence Control Systems International PTY. LTD., Chatswood, New South Wales (AU)

(21) Appl. No.: 11/718,869

(22) PCT Filed: Nov. 8, 2005
(86) PCT No.: PCT/AU05/01698

§ 371 Date: Oct. 26, 2007

(30) Foreign Application Priority Data
Nov. 8, 2004 (AU) 2004906393

(51) Int. Cl. A61N 1/05; A61N 1/36

(52) U.S. Cl. 607/40

(57) ABSTRACT

An implantable electrode arrangement (1) for stimulating excitable tissue, in particular the neosphincter to control urinary incontinence. The electrode is in the form of a extending peg with a pair of electrode elements (2, 3) extending from a base (4). The elements are arranged to fit over the tissue to be stimulated and have electrodes (5, 6) on the inner surfaces of the peg portions. The tissue located between the peg portions receives stimulation. Also disclosed are methods of treating urinary incontinence by electrically stimulating a band of smooth muscle and electrode having an insulating member and electrodes on the inner surface such that an electric field applied to tissue is confined.

The disclosure of international patent application WO01/10357 is herein incorporated in its entirety by reference.

### FIELD OF THE INVENTION

The present invention relates to an electrode arrangement for stimulating excitable tissue, and more particularly, but not exclusively, to an implantable electrode arrangement for use in treating urinary incontinence, to a method of stimulating excitable tissue and, more particularly, but not exclusively, to a method of treating urinary incontinence.

### BACKGROUND OF THE INVENTION

Electrodes for implanting into humans or animals (implantable electrodes) have been proposed for several treatments of medical conditions. The best known are probably electrodes used for cardiac stimulation (eg. in cardiac pacemaking applications). Implantable electrodes have been proposed for use in other areas, including for management of chronic pain and for management of specific types of urinary incontinence (for example, sacral nerve stimulation for urge incontinence).

Various configurations of electrodes have been proposed. These include “cuff” electrodes which are designed to wrap around nerves to electrically stimulate the nerve. Other electrode designs use “active tips” that can be placed against the excitable tissue (for example, transveous pacemaker leads which are placed in contact with the inner surface of the heart).

International patent application WO01/10357 (the disclosure of which is herein incorporated in its entirety by reference) proposes a method and an apparatus for treating incontinence in humans, which includes the steps of forming a neosphincter from smooth muscle tissue taken from elsewhere in the patient's body, and wrapping the neosphincter around the urethra. An implantable stimulator provides electrical signals to the neosphincter by way of two or more electrodes. The electrical signals stimulate the neosphincter to maintain tone about the urethra to prevent emptying of the bladder until the user wishes to urinate. The stimulator may provide a further electrical signal (or stop providing signals) to allow the neosphincter to relax and so enable the individual to urinate.

One advantage of using innervated smooth muscle is that such tissue requires only low power electrical signals to be applied to produce a continuous contraction. The implantable stimulator device may therefore operate with relatively low power Consumption to produce a superior sphincter action.

It is believed, however, that current electrode arrangements proposed for stimulation of the neosphincter in WO01/10357 could be improved upon.

### SUMMARY OF THE INVENTION

In accordance with an embodiment, the present invention provides an implantable electrode arrangement, for stimulating excitable tissue, the electrode arrangement comprising first and second electrode elements being arranged, in use, to extend with respect to each other so as to form a gap between them for receiving tissue, the first and second electrode elements including first and second electrodes respectively, the first and second electrodes being positionable, in use, in proximity to each other and being arranged, when an electrical signal is applied to them, to apply an electric field between them to stimulate the tissue between them.

In one embodiment, the first and second electrodes are positionable opposite to each other. They may be directly opposite to each other or they may be somewhat offset from opposite.

In one embodiment each electrode element has a length dimension and a width dimension, the length dimension being longer than the width dimension. The length dimension is, in one embodiment, substantially longer than the width dimension so each electrode element can be said to be elongate. The length may be greater than twice the width, and, in one embodiment may be greater than two and a half times the width. In one embodiment, the length may be greater than four times the width, in another embodiment greater than three times the width. In one embodiment, the length can be in a range of anywhere from six times the width to one and a half times the width.

The length may be governed in one embodiment by the extent of the tissue which is wished to be simulated. Where the tissue is a sphincter, such as a neosphincter as described in the above-referenced PCT application, the length of the electrode element may depend on the length of the neosphincter, and in one embodiment, the length may be the same as or greater than the neosphincter.

The electrode elements have a depth dimension that, in one embodiment, is smaller than the width dimension, so that the elements are substantially flat in profile. In one embodiment, the flatness of the electrode element facilitates avoiding irritation of tissue that may be in contact with the electrode element in use.

In one embodiment, the first and second electrode elements are mounted at proximal ends thereof to a mounting. In an embodiment, the electrode elements are in the form of fingers extending from the mounting. The mounting forms a base connecting the fingers at their proximal ends. The fingers define a gap there within which the tissue may be seated. The electrodes may be formed on inner surfaces of the fingers, in use contacting opposite sides of the tissue. The electrodes may present a substantially flat external surface to the tissue. In an embodiment, the electrode elements extend substantially parallel to each other.

In one embodiment, the surface of the electrode element contacting the tissue to be stimulated may be slightly convex in the transverse direction. A radius of curvature of the convex surface way be in a range between 1 mm and 50 mm. The convex from surface may improve contact with the tissue to be stimulated.

In an embodiment, the electrode elements include insulating material to prevent electrical stimulation being applied to tissues other than the tissue received between the electrode elements. The insulating material may be provided on the outer surface of the electrode elements, in use facing away from the excitable tissue.

In one embodiment, insulating material is also provided on the inner surface of the electrode elements. In this embodiment an opening is provided in the insulating material on the inner surface and the electrode is provided within the opening. In one embodiment, the opening is elongate, and may be in the form of a slit in the insulation.

In an embodiment, the first and second electrodes are arranged to create a confined electric field between them when an electrical signal is applied to them, in order to stimulate the tissues. In one embodiment, the electric field is confined to the extent that stimulation of tissue external to the tissue desired to be stimulated is minimised or avoided.

In an embodiment, the electrode elements are flexible. This enables them to adapt to conform with the profile of the received tissue and/or external tissue they may be seated against. It also preferably enables them to flex to accommodate profile changes which may occur in use, for example due to contraction and relaxation of the tissue between the electrode elements.

In an embodiment, the surfaces of the electrode elements are smooth so as to reduce the risk of erosion or trauma to tissue.

The excitable tissue that the electrode arrangement may stimulate may be smooth muscle tissue, innervated smooth muscle tissue, nerve tissue, or any other excitable tissue and/or contractile tissue. The tissue may be artificially produced, eg grown in an artificial medium.

In one application, the electrode arrangement is arranged for positioning about a neosphincter forming part of a system for controlling incontinence, such as described in international patent application number WO01/10357. The electrode arrangement may be implanted so that a portion of the neosphincter is received between the electrode elements. The electrode arrangement may be implanted such that the electrodes lie between 30° and 90°, preferably perpendicular to nerves within the innervated smooth muscle so that the muscle tissue receives maximum electrical field intensity between the electrodes when an electrical signal is delivered. The electrical field may then be confined between the electrodes to efficiently stimulate the neosphincter.

Although in some configurations it is mot desirable that the electrode lie at right angles to a substantial number of nerves within the innervated smooth muscle, the electrode can lie at any angle with respect to the smooth muscle and still provide benefit. The electrode/nerve angle may vary in one embodiment between 60° either side of perpendicular. In one embodiment the angle is in a range 40° either side of the perpendicular. In another embodiment the angle is in a range 30° either side of the perpendicular and in another embodiment 20° either side of the perpendicular.

In one embodiment, the electrode arrangement of the present invention has the advantage of being suited for stimulation of innervated smooth muscle tissue so that low stimulus currents are required to stimulate nerves running close to the surface of the smooth muscle tissue. A further advantage of some embodiments is that the electrodes intimately contact the surface of the smooth muscle without causing mechanical damage to the tissue (erosion). Further, the electrode elements may be built in a range of varying lengths/mechanical configurations to deal with a range of smooth muscle sizes which may be required in a range of applications to treat medical conditions (fecal incontinence, gastric reflux, heart conditions etc).

It is a particular advantage of at least embodiments of the electrode arrangement of the present invention that they are suited for use in the stimulation of tissues forming sphincters, such as the neosphincter described in the above-referenced PCT application, and other sphincters which way be used for treating other conditions, such as fecal incontinence, gastric reflux, cardiac conditions etc.

The electrode arrangement of the present invention is not limited to application in controlling urinary incontinence. It may be suited to other application, in particular, applications for stimulating contractile tissue, in particular applications in which nerves within a smooth muscle organs or the smooth muscle itself is required to be stimulated, in a person or animal.

In accordance with a further embodiment, the present invention provides an electrode arrangement for controlling urinary incontinence, including an electrode arrangement in accordance with the first aspect of the present invention, dimensioned to be implantable in a patient to stimulate contractile tissue to control urinary incontinence.

In accordance with a further embodiment, the present invention provides a method of stimulating excitable tissue, comprising the steps of operating an electrode arrangement in accordance with the first aspect of the invention, wherein excitable tissue lies between the electrode elements, and app lying a electrical stimulus to the electrodes so as to create a confined electric field between the electrodes to stimulate the tissue.

In at embodiment, the tissue is a muscle neosphincter for controlling incontinence, as discussed in WO01/10357.

In accordance with a further embodiment, the present invention provides a method of surgically implanting an electrode arrangement such as the electrode arrangement discussed above, comprising the steps of implanting the electrode arrangement in a patient so that tissue to be stimulated is received between the first and second electrode elements.

In an embodiment, the step of securing includes the step of fixing distal ends of the electrode elements together.

In accordance with a further embodiment, the present invention provides a method of treating urinary incontinence, comprising the steps of surgically implanting an electrode arrangement as discussed above so that the electrode elements extend about a portion of a contractile tissue sphincter arranged about the urethra, and providing electrical signals to the electrodes causing the sphincter to contract and maintain tone preventing urine from passing through the urethra.

In an embodiment, the step of implanting the electrode arrangement includes the step of implanting the electrode arrangement so that the electrodes run at an angle to the nerves within the tissue. In an embodiment, the electrodes run at an angle of between 30° and 150° to the nerves within the tissue. In an embodiment, the angle is 40° to 140°. In a further embodiment the angle is 50° to 130°. In a further embodiment the angle is 60° to 120°. In a further embodiment the angle is 70° to 110°. In a further embodiment the angle is 80° to 100°. In a further embodiment the angle is substantially 90°.

In accordance with a further embodiment, the present invention provides a method of treating urinary incontinence, comprising the steps of electrically stimulating a smooth muscle sphincter arranged about the urethra, the step of electrically stimulating including electrically stimulating a band of muscle of the smooth muscle sphincter, the band extending across the width of the smooth muscle sphincter.

The band preferably extends in a direction which is between 30° and 150° angle to nerves running within the smooth muscle sphincter. In an embodiment the angle is 40° to 140°. In a further embodiment the angle is 50° to 130°. In a further embodiment the angle is 60° to 120°. In a further embodiment the angle is 70° to 110°. In a further embodiment the angle is 80° to 100°. In a further embodiment the angle is substantially 90°.

In accordance with a further embodiment, the present invention provides a method of controlling innervated smooth muscle tissue comprising the steps of electrically stimulating a portion of the smooth muscle tissue in the form of a band extending in the smooth muscle tissue.

In an embodiment, the band extends at an angle of between 30° and 150° to nerves running within the smooth muscle. In an embodiment, the angle is 40° to 140°. In a further embodiment the angle is 50° to 130°. In a further embodiment the angle is 60° to 120°. In a further embodiment the angle is 70° to 110°. In a further embodiment the angle is 80° to 100°. In a further embodiment the angle is substantially 90°.

In accordance with a further embodiment, the present invention provides an apparatus for stimulating human or animal excitable tissue, the apparatus comprising an implantable electrode arrangement as discussed above, and a stimulator, the stimulator being arranged to provide signals to the first and second electrodes for stimulation of the excitable tissue.

In one embodiment, the apparatus further comprises a stimulator controller, the stimulator controller arranged to be operable to control the stimulation signals provided by the stimulator.

In one embodiment, the apparatus further comprises a stimulator programmer, the stimulator programmer being arranged to program control parameters of the stimulator.

In the above aspects of the present invention, the electrode arrangement includes a pair of electrode elements which, in one embodiment, are mounted together by a mounting at proximal ends thereof. In a further embodiment, the present invention may include a single electrode element which may be used on its own or together with other similar electrode elements in the form of a “set”, to provide appropriate stimulation to, for example, contractile tissue.

In accordance with a further embodiment, the present invention provides an electrode element for stimulating excitable tissue, the electrode element comprising an insulating member having an inner surface, at least one active electrode being provided on the inner surface, the electrode element being positionable adjacent tissue to be stimulated and the electrode being arranged to provide a electric field to stimulate the tissue, when a signal is applied to the electrode.

In an embodiment, the electrode is arranged to create a confined electric field, to eliminate or limit stimulation to tissues adjacent to the tissue which it is desired to stimulate.

A single electrode element may be used to stimulate tissue appropriately, or may be used with other similar electrode elements in the form of a “set” to stimulate tissue.

In accordance with a further embodiment, the present invention provides a set of electrode elements, including a plurality of electrode elements in accordance with the ninth aspect of the present invention, the set being arranged so that at least a pair of the electrode elements can be placed in proximity to each other to enable the respective electrodes to create a confined electric field between them across excitable tissue to be stimulated.

In an embodiment, the electrode is arranged to create a confined electric field, to eliminate or limit stimulation to tissues adjacent to the tissue which it is desired to stimulate.

The electrode elements may be arranged to be placed substantially opposite each other.

In accordance with a further embodiment, the present invention provides a method of treating a disorder in a patient, comprising the steps of utilising the electrode arrangement as discussed above, in order to stimulate contractile tissue in the patient to treat the disorder.

In accordance with a further embodiment, the present invention provides an electrode apparatus for controlling a disorder in a patient, the electrode apparatus comprising an electrode arrangement as discussed above, arranged for the stimulation of contractile tissue to treat the disorder.

In accordance with a further embodiment, the present invention provides an apparatus for stimulating excitable tissue, the apparatus comprising an implantable electrode element as discussed above, and a stimulator, the stimulator being arranged to provide signals to the electrode for stimulation of the excitable tissue.

In accordance with a further embodiment, the present invention provides a method of treating a disorder in a patient, comprising the steps of utilising an electrode element as discussed above, in order to stimulate contractile tissue in the patient to treat the disorder.

In accordance with a further embodiment, the present invention provides an electrode apparatus for controlling a disorder in the patient, the electrode apparatus comprising an electrode element as discussed above, arranged to stimulate contractile tissue to treat the disorder.

In accordance with a further embodiment, the present invention provides a method of treating a disorder in a patient, comprising the steps of stimulating contractile tissue in the patient to treat the disorder, the step of stimulating contractile tissue including stimulation of a band of the contractile tissue.

In accordance with a further embodiment, the present invention provides an electrode arrangement arranged to treat a disorder in a patient by the stimulation of contractile tissue, the electrode arrangement including stimulation means for stimulating a band of the contractile tissue.

The different embodiments of the present invention disclosed above may be combined in different combinations and with other aspects or features of the present invention and still fall within the scope of the disclosed invention. The above embodiments should not read as limiting the inventions disclosed in this application.

### BRIEF DESCRIPTION OF THE DRAWINGS

Features and advantages of the present invention will become apparent from the following description of embodiments thereof, by way of example only, with reference to the accompanying drawings, in which;

FIGS. 1a, b, c, d are isometric views of different embodiments of electrode arrangements in accordance with the present invention;

FIGS. 2a, b, c, d are side views of the embodiments of FIG. 1a, b, c, d;

FIG. 3 is a schematic diagram showing an electrode arrangement in accordance with an embodiment of the present invention in situ together with muscle tissue.

FIG. 4 is a diagram of an electrode arrangement in accordance with an embodiment of the present invention in situ together with a neosphincter for controlling urinary incontinence;

FIG. 5 is a further diagram showing an alternative in situ arrangement of an electrode arrangement in accordance with embodiment of the present invention with a neosphincter for treating urinary incontinence;

FIG. 6 is a schematic view of an electrode arrangement in accordance with a further embodiment of the present invention;

FIGS. 6, 7 and 8 are exploded perspective, plan and side views, respectively, of an electrode arrangement in accordance with a further embodiment of the invention;

FIGS. 9, 10, 11, 12 and 13 are perspective, plan, side section, plan section and detail views of a shroud component of the electrode arrangement of FIGS. 6, 7 and 8;

FIGS. 14, 15, 16, 17, 18 are perspective, rear, plan section, side section and plan views of a cover component of the electrode arrangement of FIGS. 6, 7 and 8;

FIG. 19 is a perspective view from above and one side of an electrode element in accordance with an embodiment of the present invention; and

FIG. 20 is a perspective view from above and one side of an electrode element in accordance with a further embodiment of the present invention.

### DETAILED DESCRIPTION OF EMBODIMENTS

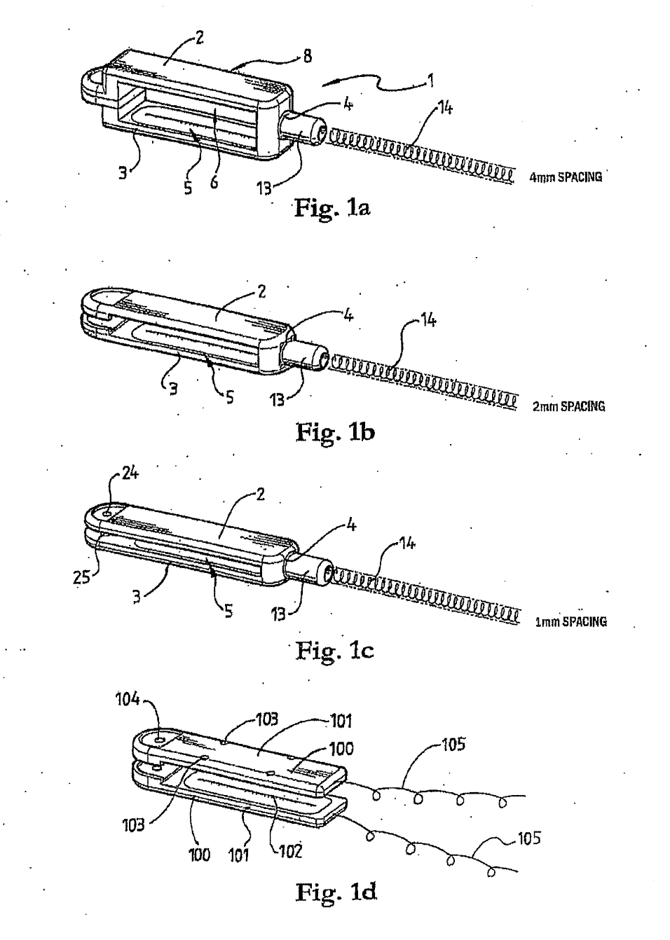

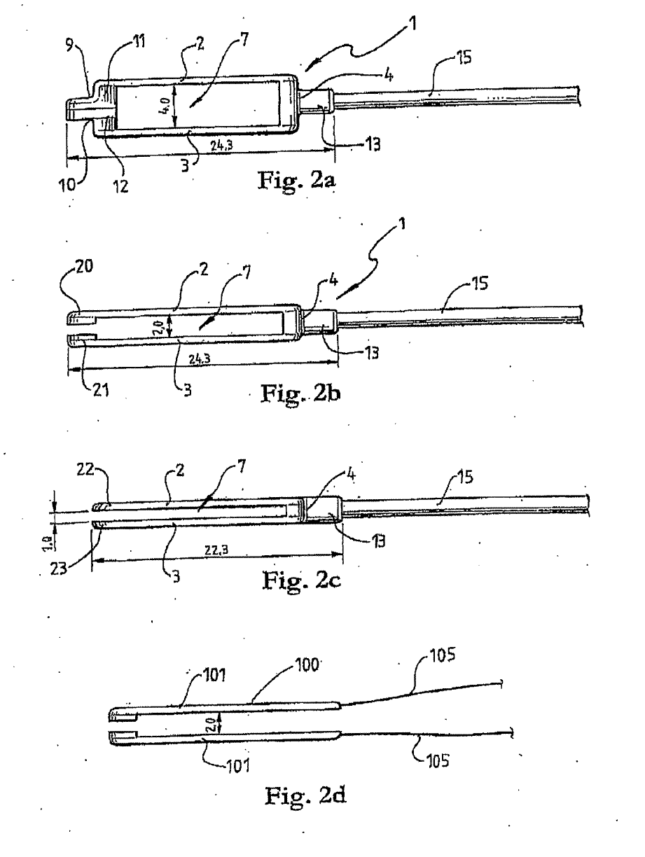

Referring to FIGS. 1a and 2a, an electrode arrangement 1 in accordance with an embodiment of the present invention is illustrated. The electrode arrangement 1 includes first and second electrode elements 2, 3, which in this example are in the form of elongate rectangular elements which extend from a mounting 4. Mounting 4 acts to secure the electrode elements 2, 3 at their proximal ends.

The electrode arrangement 1 includes electrodes 5, 6, in this embodiment being in the form of conductive plates which extend along the inside of the electrode elements 2, 3 opposite to each other. The electrodes 5, 6, may alternatively be in the form of a printed conductive medium printed on the inside surface of the electrode elements 5, 6.

The electrode elements 2, 3 are arranged so that tissue from a human or animal body may be received therebetween within the gap 7 (FIG. 2), so tissue is “sandwiched” between the electrode 5, 6 exposed surfaces.

In this embodiment, the electrode element 2, 3 are comprised mainly of insulating material 8 so that the outer surface (in this embodiment all surfaces apart from conductive electrodes 5 and 6) are insulated and do not Conduct electricity.

In this embodiment the inner surface is also insulating material with an elongate Opening in the form of a slit. The electrodes are provided at the slit.

As is most clearly shown in FIG. 2a, distal ends 9, 10 of the electrode elements 2, 3 include projecting portions 11, 12 which project inwardly from the electrode elements 2, 3 so that they meet each other. In operation, the ends 9, 10 may be secured together so that the electrode arrangement is firmly secured about the tissue which lies in the gap 7 between the electrode elements 2, 3.

In this embodiment, mounting 4 includes a strain relief member 13 for receiving an electrical conductor 14 within a cable 15 (the cable being insulated), the electrical conductor 14 being arranged for electrical connection between electrodes 5 and 6 and a device (not shown) for providing electrical signals to the electrodes 5, 6. The distal ends of the electrode elements may be fixed (if required to be fixed) by a number of means, including suture holes, press studs or any other arrangement that may not require much surgical access to “lock” the electrode elements closed. Note also, it is not essential for all embodiments that the electrode elements be fixed together at their distal ends.

In embodiments, insulating parts of the electrode elements may be composed of two sheets of bio compatible material (e.g. silicone)—which acts as an insulator, and surrounds and limits the exposed surface of thin flexible platinum foils that forms the electrodes. The silicone may be reinforced with bio compatible mesh (eg a PET or PTFE —like material), so that sutures will not tear through the silicone.

In this embodiment, the electrode elements 2, 3 are arranged to flex such that the arrangement is arranged to conform, at least to some limited extent, with the profile of the received tissue and/or external tissue that they may be seated against. The electrode elements 2, 3, are arranged to flex to conform with any changes in the profile of the received tissue which may be due to electrical stimulation.

In this embodiment, the electrode elements may be sufficiently flexible so that they conform with any changes in the profile of the received tissue and also with the profile of external tissue so they do not irritate or erode the external tissue or receive tissue. This has the advantage of increasing the lifetime of the implant.

The electrode elements may be semi-Flexible or in another embodiment totally flexible.

In a further embodiment, electrodes elements are not flexible (non flexible electrode elements are within the scope of the present intention).

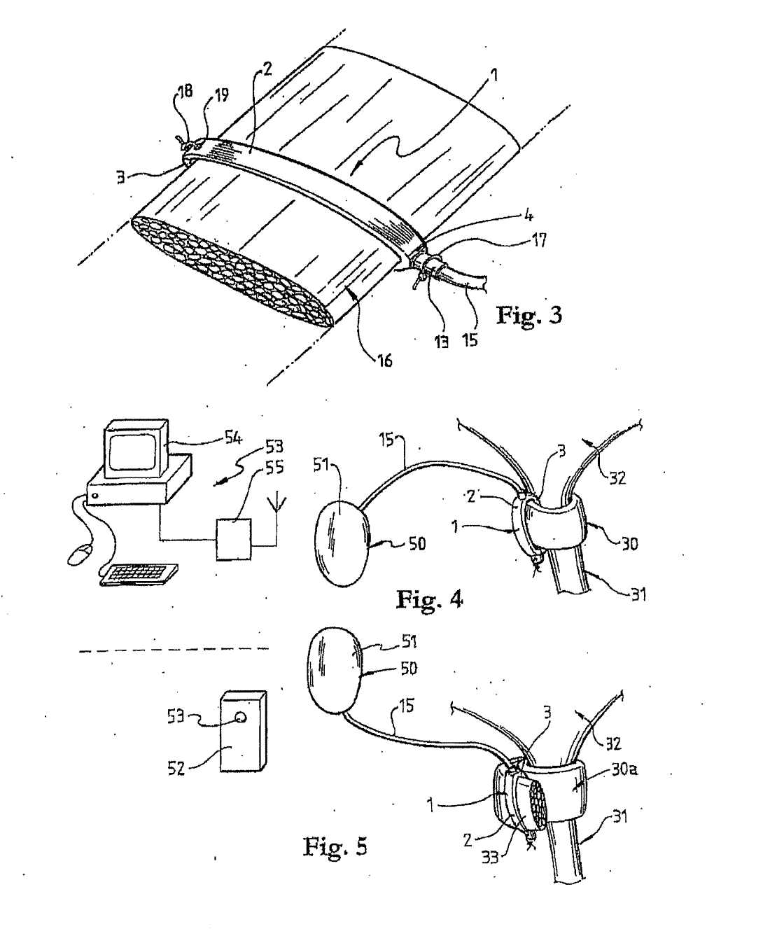

FIG. 3 schematically illustrates an electrode in accordance with the present invention in situ about muscle tissue. The same reference numerals have been used in FIG. 3 as in FIG. 1a, to designate corresponding components.

It can be seen from FIG. 3 that the muscle tissue 16 is received between electrode elements 2, 3. The electrodes 5, 6 on the inside surfaces of the electrode elements 2, 3 therefore contact the muscle tissue 16. Electrical signals may be applied to the electrodes 5, 6 resulting in an electrical field applied across the muscle tissue 16, the electrical field being confined by the electrodes and the electrode elements 2, 3 to the muscle tissue that sits between the electrode elements 2, 3. This creates a band within the smooth muscle in which the nerves are excited—causing the neo-sphincter to contract. Because the outer surfaces of the electrode elements 2, 3 are of insulating material, adjacent tissue will not be stimulated.

Tethers 17 and 18 may be used to hold the electrode arrangement 1 in place to adjacent tissue within the human or animal body.

Note that in the embodiment of FIG. 3 holes 19 in the electrode element 2 and also (not shown) electrode element 3 are provided to receive the tether in the distal ends of the electrode elements 2, 3 and also tie those ends together. Although the holes were not shown in the embodiment of FIGS. 1a and 2a, they may be provided for this purpose.

FIG. 3 also illustrates how the electrode arrangement 1 “flexes” to conform with the surface profile of the muscle tissue 16.

In the embodiment of FIG. 1a, electrode elements 2, 3 are in the form of fingers extending from a base formed by the mounting 4. The tap 7 is defined between the fingers, 2, 3, for receiving muscle tissue. The gap has no sides as there are no side walls extending between the fingers 2, 3. This enables the arrangement to be slid over the muscle tissue from one end, in an analogous manner to a clothes peg over clothes. This advantageously facilitates implantation of the electrode arrangement. This can facilitate the type of surgery and speed with which the Surgery is carried out.

One application of the embodiment of FIG. 1a is in stimulation of a neosphincter such as used in the method of treating incontinence disclosed in WO01/10357. Referring to FIG. 3, muscle tissue 16 in such an application would be a portion of the neosphincter surrounding the urethra. In the application disclosed in WO01/10357, the neosphincter is innervated smooth muscle, and the nerves run coincident with the length of the muscle 16. The electrode elements 2, 3, run substantially at right angles to the direction of the nerves and the confined electrical field therefore provides optimum stimulation. Note that the electrode elements may not run precisely at right angles to the direction of the nerves, but may run within an angular range of 90° to the angles of the nerves. In one embodiment the angular range may be 80° to 100° of the majority of the nerves running in the smooth muscle sphincter. In another embodiment, the angle may be 70° to 110°. In another embodiment the range is 40° to 140°. In a further embodiment the range is 50° to 130°. In a further embodiment the range is 60° to 120°. In a further embodiment the angle is substantially 90°.

The embodiment of FIG. 1a is sized for the neosphincter application. Dimensions are given in the drawings in millimetres (FIG. 2a). The gap 7 is approximately 4 millimetres, and length of the electrode arrangement is 24.3 millimeters.

Other embodiments are shown in FIGS. 1b and c and 2b and c. The same reference numerals as used in FIGS. 1 and 2a have been used to designate corresponding components and no further description will be given of these components. The embodiments of FIGS. 1 and 2b and c are dimensioned to be suitable for neosphincters over a range of thicknesses. As far as this may enable the electrode arrangement to deal with a number of different sizes of neosphincters which may be used for a number of different applications other than urinary incontinence e.g. esophageal reflux, treatment of cardiac conditions, fecal incontinence etc. (see later).

In the embodiment of FIG. 1b, the distal ends 20, 21 include shallower projections than the embodiment of FIG. 1a.

In the embodiment of FIG. 1c, the distal ends 22, 23, do not have any projections, as the dimension of the gap 7 is small enough not to require them (with the flexing provided by the electrode elements 2,3). The distal ends 22, 23 are provided with holes 24, 25 facilitating securing by way of a tether (as in FIG. 3).

FIG. 4 is a diagram showing an electrode arrangement 1 on a smooth muscle neosphincter 30. In accordance with the application disclosed in WO 01/10357 the neosphincter 30 is shown wrapped around the urethra 31. The bladder 32 is illustrated positioned just above the neosphincter 30. The electrode elements 2, 3 receive between them a band of neosphincter 30. Electrodes 5, 6 (not shown in FIG. 4) contact the band of muscle tissue. Nerves in the innervated neosphincter 30 run in a direction which is substantially at right angles to the electrode elements 2, 3.

FIG. 5 shows an alternative arrangement, where the neosphincter 30a in this alternative includes an overlapping portion 33 which overlaps the neosphincter 30a. The electrode arrangement 1 in this alternative is positioned so that the electrode elements 2, 3 extend about, a band of the overlapping portion 33.

In the arrangement of FIG. 4 an outer surface of electrode element 3 is in contact with the urethra and the bladder neck. In the alternative of FIG. 5, the outer surface of the electrode element 3 is in contact with outer surface of the neosphincter and does not contact the urethra or bladder neck.

In both the arrangement of FIGS. 4 and 5, the electrode arrangements 1 are shown connected via lead 15 to a stimulator 50. The stimulator 50 includes electronic circuitry arranged to generate signals for transmission to the electrodes and application to the smooth muscle of the sphincter 30, 30a. As described in WO 014/10357, the stimulators so include a biocompatible housing 51 mounting the electrical circuitry (not shown).

Also shown schematically adjacent to FIGS. 4 and 5 are a stimulator controller 52 and a stimulator programmer system 53. The stimulator controller 52 includes user operable means 53 (in this case a button) which, when actuated, causes a signal to be transmitted from the stimulator controller 52 to the stimulator 50, to effect the operation of the stimulator 50. For example, if a patient wishes to urinate, they may actuate the controller 52 to cause the stimulator 50 to stop sending stimulation signals to the electrode arrangement, in order to enable the muscle sphincter to relax and the patient to be able to urinate.

The stimulator programmer unit 53 in this embodiment includes a computing system 54 (represented in the drawing an a conventional PC, but may be any type of computing system) and a transmitter 55 arranged to transmit instructions from the computing system 54 to the stimulator 50. The stimulator programmer 53 is used to program and calibrate the stimulator 50 for optimal operation. It may also receive stimulated telemetry information indicative of one or more parameters of the stimulator, for monitoring by an operative.

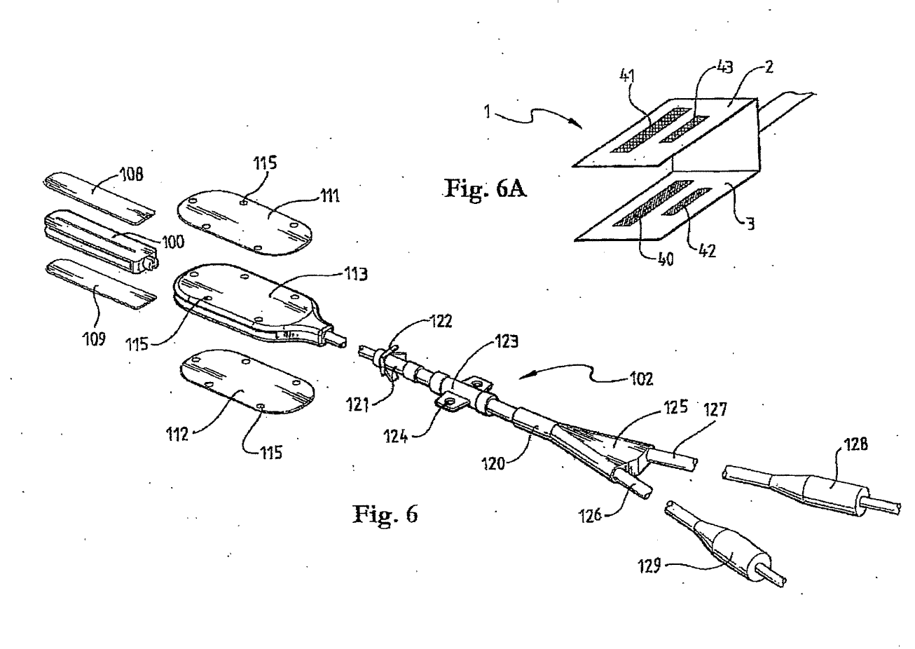

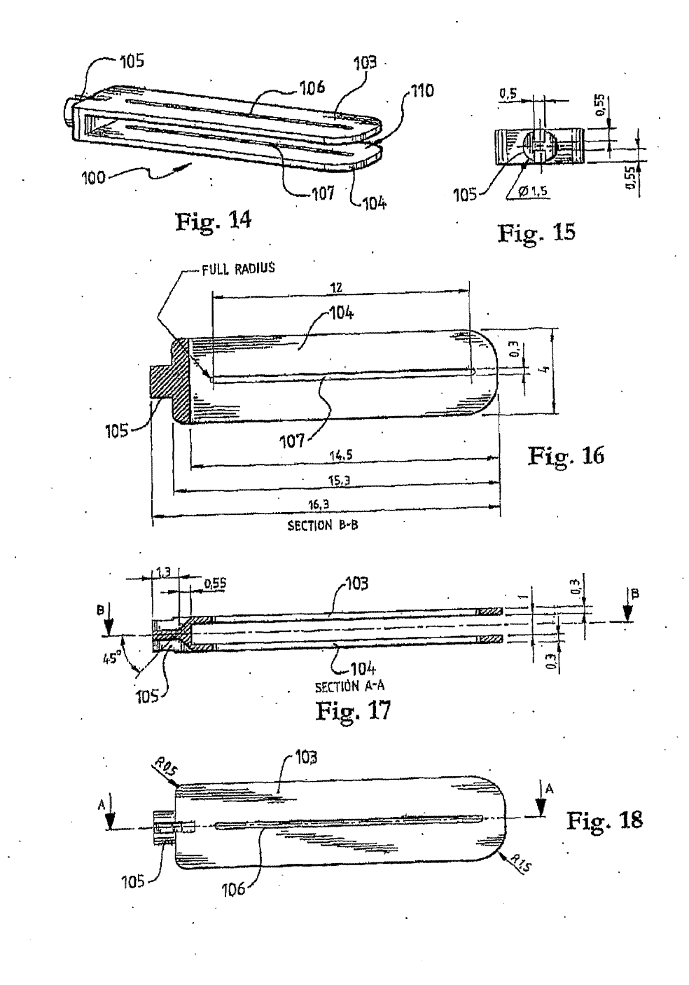

A further embodiment of a electrode arrangement in accordance with the present invention will now be described with reference to FIGS. 6 through 18.

The electrode arrangement of this embodiment comprises a number of components. These include an electrode cover 100 (shown in most detail in FIGS. 13 through 17).

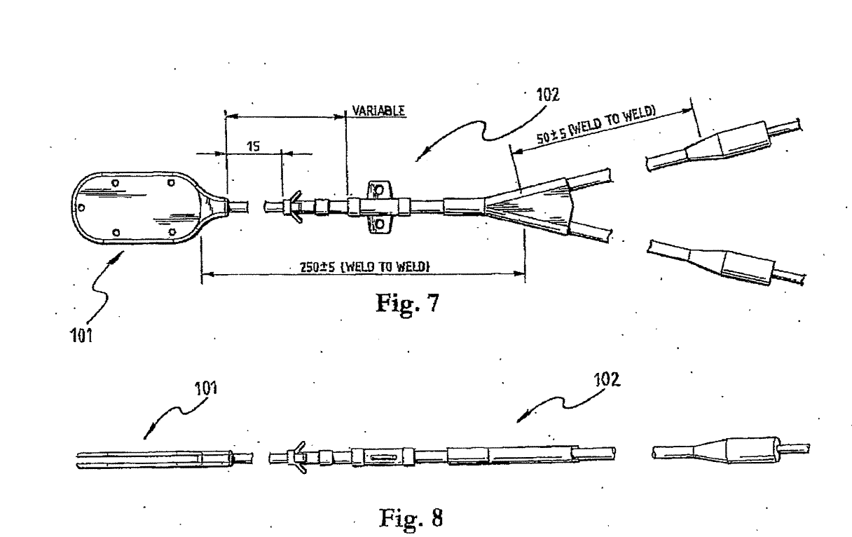

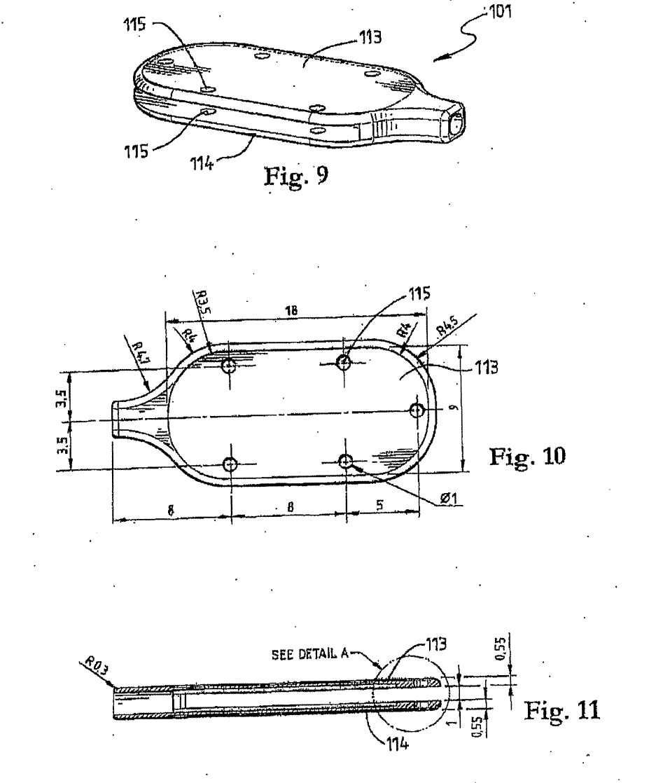

The components also include an electrode shroud (shown in best detail in FIGS. 9 through 12) and also an electrode lead 102 (shown in FIGS. 6, 7 & 8, together with the other components of the electrode arrangement).

In this embodiment the first and second electrode elements are formed by the electrode cover 100, which includes insulating elements 103, 104 extending from a base 105. The insulating extending elements 103, 104 are formed with a slot 106, 107, respectively, extending substantially along the length of the extending elements 103, 104. When the electrode arrangement is assembled, platinum foil electrodes 108, 109 (FIG. 6) a placed on the outer surfaces of the elements of the elements 103, 104 so that they are insulated from the gap 110 formed between the elements 103, 104 apart from the slots 106, 107, which expose portions of the conductive plates 108, 109 to the gap 110 (and, in use, to any tissue seated within the gap).

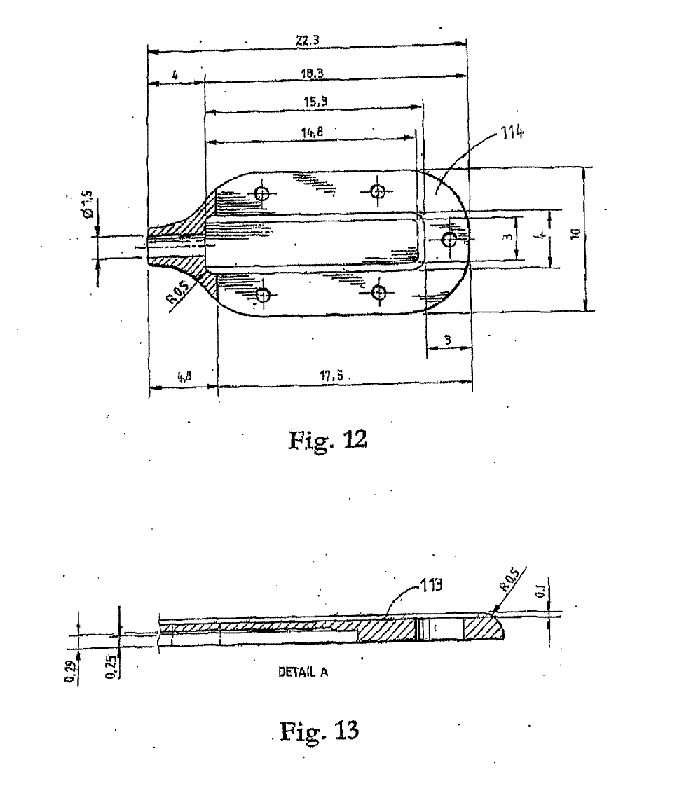

When assembled, the electrode cover 100 and platinum electrode foils 108, 209 seat within the electrode shroud 101 as best shown in FIGS. 9, 10, 11 & 12. FIG. 12 in particular shown in in-section where the electrode cover seats.

Electrode shroud 1 is formed from silicone. In order to provide reinforcement, PET mesh covers 111, 112 are provided to fit to upper 113 and lower 114 extending portions of the shroud 101. Suture holes 115, 116 are provided in the covers 111, 112 and also in the elements 113, 114 of the shroud 101. Note that the reinforcement can be provided by other means and is not limited to PET mesh. Further, the electrode shroud need not be in silicone but could be of other bio-compatible material and may not require re-inforcement. Further, note that other means for affixing to the tissue may be provided other than suture holes or instead of suture holes.

The electrode lead 102 is a multi-component arrangement which includes an outer insulating cover 120, a tine collar 121 including tines 122 for retaining the lead in position within a patient. It also includes a sutured collar 123 including suture holes 124 for suturing to patient tissue to also facilitate retaining the lead 102 in position. There is also bifurcation moulding 125 which enables the lead to split into two parts 126, 127, which may contain separate conductors, and connectors 128, 129 which may be arranged to contact to a simulation device.

In the above embodiments, the electrode arrangement includes a pair of electrode elements which extend away from a base which joins them together at their proximal ends. In a further embodiment, a single electrode element which is not joined at any base is provided. This single electrode element may be used to provide stimulation to contractile tissue on its own, or may be used together with one or more similar electrode elements to provide stimulation.

Referring to FIG. 1d and FIG. 2d, a pair of electrode elements in accordance with this embodiment are illustrated. The electrode element 100, comprises elongate insulating member 101 which mounts on its inner surface an electrode 102, in this embodiment being in the form of a long thin line extending substantially the majority of the length of the electrode element 100. Suture holes 103 are provided to enable the electrode element 100 to be secured to tissue. A further suture hole 104 is provided at one end of the electrode element 100 in order to enable it to be secured to a similar end of a similar electrode element 100. Holes may also be provided for this purpose at the opposite end (not shown in the drawings). A lead 105 extends from the electrode element 100 to a stimulator (not shown). In FIG. 1 there are two electrode elements 100 shown. In such an arrangement they may be used as a “set”, for example secured either side of a contractile tissue sphincter, operating in a similar manner to the embodiments of FIG. 1, a, b, c.

In the above described embodiments, each electrode element is provided with a single electrode. The single electrode is an elongate electrode extending substantially the majority of the length of the electrode element.

One advantage of having thin electrodes bounded by insulating material on either side is that the arrangement operates to confine the electric field produced by the electrode to the tissue immediately adjacent the electrode. This reduces or prevents stimulation of tissue that it is not desirable to stimulate eg. tissue external to a contractile tissue sphincter being controlled.

The extent of confinement of the electric field may depend upon the application to which the electrode is being put. It is desirable that confinement be sufficient to prevent or substantially avoid stimulation of tissues other than the tissue for which stimulation is required e.g. it should avoid stimulation of external tissues.

The present invention is not limited to the electrode element having a single electrode, however. There may be multiple electrodes, including electrodes side by side or electrodes arranged in an array. Further, the electrodes need not be “thin line” shaped electrodes, but may be any shape, including square, rectangular, round, etc.

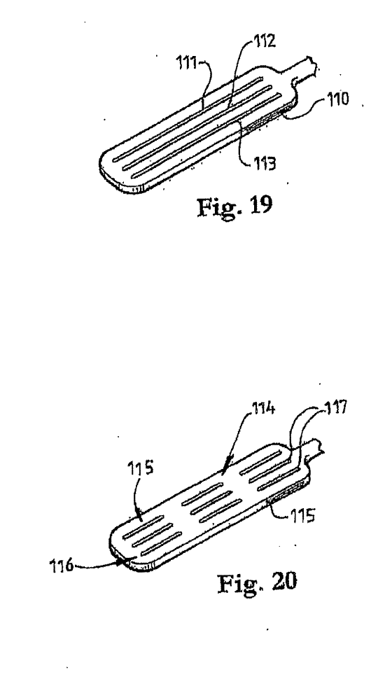

FIG. 19 is an embodiment of an electrode arrangement in accordance with the present invention which includes an electrode element 110 formed of insulating material and being provided with three electrodes 110, 112 and 113. These are “thin line” electrodes, similar to the electrodes of previous embodiments. In this case, however, electrode 112 is the active electrode and electrodes 111 and 110 form “shield” electrodes. This facilitates confinement of the electrical field provided by electrode 112 to the tissue which it is desired to stimulate.

FIG. 20 is an embodiment of an electrode arrangement in accordance with the present invention which includes an array of electrodes 114. The array of electrodes 114 is provided on an electrode element 115 similar to that described in previous embodiments. In this example, the array of electrodes includes a row 115 of three electrodes and a column 116 of three electrodes. The column can be considered to be a single “thin line” electrode broken up into three separate electrodes. The ends 117 of the electrodes provide field concentration points providing better current density and concentration and this may facilitate lower currents for appropriate stimulation.

In the above-described embodiments the electrode elements are substantially rectangular in form. They may be other shapes than rectangular eg cylindrical, rounded.

The electrode arrangement of the present invention is not limited to application in incontinence control in accordance with the system of WO01/10357. The electrode arrangement may be used for stimulating smooth muscle in other incontinence control systems, or other applications in which nerves within smooth muscle organs or the smooth muscle itself is required to be stimulated. The dimensions (eg. particularly the length of the electrode elements and the dimensions of the gap between them, and also there widths) may be varied according to the application and are not limited to the dimensions disclosed in relation to the specifically described embodiments.

Other applications for which an electrode arrangement in accordance with the present invention may be useful include pacing the stomach and other organs, for treating gastro-esophageal reflux problems and fecal incontinence.

The electrode arrangement in accordance with the present invention may be used to simulate any excitable tissue. In particular, it may be utilized to stimulate contractile tissue for applications such as fecal incontinence, reflux problems, cardiac disorders, and any other disorder where the use of contractile tissue, such as a contractile tissue sphincter, for example, may be useful.

In the embodiments described above, a lead 15 is provided housing a conductor 14 for conducting electrical signals to the electrodes. In an alternative embodiment, a signal generator may be housed in a body which is adjacent to or part of the electrode arrangement, obviating the need for a lead to connect to a stimulator implanted elsewhere in the body.

The electrode arrangements of the described embodiments are bipolar configurations. The electrode arrangements of the present invention are not limited to bipolar configurations, and electrode arrangements including tripolar and multi-polar configurations may be utilised. A tripolar or multi-polar arrangement may be used to stimulate different muscle nerve areas, and/or for sensing purposes to, for example, provide feedback on the effects of stimulation. Tripolar or multi-polar arrangements may be formed by forming separated conductive plates on the electrode elements, Another alternative is to provide more than two electrode elements. For example, three or four electrode elements could be provided in the form of three or more fingers extending from the mounting 4.

In yet a further embodiment, which may be suited for applications with tissues and sphincters for treating urinary incontinence (such as disclosed in the above-referenced PCT application), fecal incontinence, gastro-esophageal reflux problem and cardiac conditions etc, a monopolar electrode may be provided. The monopolar electrode may be in the form of a single electrode on a single electrode element such as described above. The electrode may be a single point electrode or a thin line electrode provided by current density, of relatively high current density to the edges. Current focus may be provided by a further return electrode placed in another part of the patient.

In yet a further embodiment, a point electrode may be placed on the inner surface of the smooth muscle (or other contractile tissue) and the return electrode in another part of the body.

FIG. 6A is a schematic diagram of an electrode arrangement in accordance with a further embodiment of the present invention. The schematic diagram illustrates multi-polar electrodes on the inner surfaces of electrode elements 2, 3. The electrodes include stimulating electrodes 40, 41 and sensing electrodes 42, 43.

A further alternative electrode, as discussed above, may include a monopolar arrangement including a single electrode placed on the elements described in relation to FIGS. 19 and 20.

In the treatment of a cardiac condition, an electrode arrangement in accordance with an embodiment of the prevent invention may be used in a counter-pulsation application. In counter-pulsation, a contractile tissue sphincter is placed on the ascending aorta and stimulated in order to facilitate blood flow, particularly for conditions such as angina and congestive heart failure.

An electrode arrangement of the present invention may also be utilised in stimulation of tissues that are not in a patient but may be, for example, for the purpose of evaluating physiological properties of such tissues or for using them as test systems for the development of therapeutic agents or techniques. Electrodes of the present invention may be useful for such in vitro techniques.

Some of the embodiments of the electrode arrangement described above include extending electrode elements joined at one end to a mounting Embodiments (described above) are envisaged where the electrode elements may not be joined at a mounting but may, instead, be separated. In use, they may be separately fixed (eg sutured) to the tissue so that they are opposite each other and function to provide the appropriate electric field.

1. An implantable electrode arrangement, for stimulating contractile tissue, the electrode arrangement comprising first and second electrode elements being arranged, in use, to extend with respect to each other so as to form a gap between them arranged for receiving contractile tissue, the first and second electrode elements including first and second electrodes respectively, the first and second electrodes being positionable, in use, in proximity to each other and being arranged, when an electrical signal is applied to them, to apply an electric field between them to stimulate the contractile tissue between them.

2. An implantable electrode arrangement in accordance with claim 1, the first and second electrodes being positionable, in use, opposite to each other.

3. An electrode arrangement in accordance with claim 1, wherein each electrode element has a length dimension and a width dimension, and the length dimension is substantially longer than the width dimension, so that the electrode elements are of elongate form.

4-5. (canceled)

6. An electrode arrangement in accordance with claim 3, wherein a depth dimension of the electrode elements is smaller than the width dimension.

7. An electrode arrangement in accordance with claim 6, the electrode elements being substantially flat in profile.

8. An electrode arrangement in accordance with claim 1, wherein the first and second electrodes are provided on inner surfaces of the first and second electrode elements, in use being arranged to contract opposite parts of the tissue so that a portion of the tissue lies between them.

9-28. (canceled)

29. An electrode arrangement in accordance with claim 1, the first and second electrode elements being mounted at proximal ends thereto to a mounting, from which they extend with respect to each other so as to form a gap between them for receiving the tissue.

30. An electrode arrangement in accordance with claim 29, the electrode elements being in the form of fingers extending from the mounting, the mounting forming a base connecting the fingers at their proximal ends.

31. An electrode arrangement in accordance with claim 30, wherein the gap is open on all sides apart from where the electrode elements are joined at the base, so that the arrangement may be placed over the tissue from one end, in an analogous manner to a peg.

32. An electrode arrangement in accordance with claim 1, wherein the electrode elements are flexible, whereby to enable them to adapt to the shape of the received tissue and/or external tissue that they may be seated against in use.

33. An electrode arrangement in accordance with claim 1, the first and second electrodes being arranged to create a confined electric field between them when an electrical signal is applied to them, in order to stimulate the tissue.

34-38. (canceled)

39. An electrode arrangement for controlling urinary incontinence, including an electrode arrangement in accordance with claim 1, arranged to be implantable in a patient to stimulate contractile tissue to control urinary incontinence.

40. An electrode arrangement in accordance with claim 39, being arranged such that the first and second electrode elements are positionable either side of a band of tissue forming a portion of neosphincter wrapped around the urethra, the neosphincter being arranged to contract on stimulation by the electrodes, in order to maintain continence.

41. A method of stimulating contractile tissue, comprising the steps of operating an electrode arrangement in accordance with claim 1, wherein contractile tissue lies between the electrode elements, and applying an electrical stimulus to the electrodes to create a confined electric field between the electrodes to stimulate the tissue.

42. A method in accordance with claim 41, wherein the contractile tissue is formed into a neosphincter about the patient's urethra, for controlling urinary incontinence.

43-46. (canceled)

47. A method of treating urinary incontinence, comprising the steps of surgically implanting an electrode arrangement in accordance with claim 1, so that the electrode elements extend about a portion of a contractile tissue sphincter arranged about the urethra, and providing electrical signals to the electrodes causing the sphincter to contract and prevent urine from passing through the urethra.

48. A method in accordance with claim 47, wherein the contractible tissue is smooth muscle.

49. A method in accordance with claim 48, wherein the step of implanting the electrode arrangement includes the step of implanting the electrode arrangement so that the electrodes run substantially at right angles to the nerves of the smooth muscle tissue.

50. A method of treating urinary incontinence, comprising the steps of electrically stimulating a smooth muscle sphincter arranged about the urethra, the step of electrically stimulating including electrically stimulating a band of muscle of the smooth muscle sphincter.

51-82. (canceled)

83. An apparatus for stimulating excitable tissue, the apparatus comprising an implantable electrode arrangement in accordance with claim 1, and a stimulator, the stimulator being arranged to provide signals to the first and second electrodes for stimulation of the contractile tissue.

84. An apparatus in accordance with claim 83, further comprising a stimulator controller, the stimulator controller arranged to be operable to control the stimulation signals provided by the stimulator.

85. An apparatus in accordance with claim 83, further comprising a stimulator programmer, the stimulator programmer being arranged to program control parameters of the stimulator.

86-129. (canceled)
